(19) United States
(12) Patent Application Publication
(10) Pub. No.: US 2002/0072798 A1
(43) Pub. Date: Jun. 13, 2002

(54) Muscle tissue engineering

(76) Inventors: Jens Uwe Riesle, Amsterdam (NL); Clemens Antoni van Blitterswijk, Hekendorp (NL); Maria Papadaki, Cambridge, MA (US); Robert Langer, Newton, MA (US)

Correspondence Address: John P. Iwanicki, BANNER & WITCOFF, LTD., 28 State Street, 28th Floor, Boston, MA, 02109, US

(21) Appl. No.: 09/955,733

(22) Filed: Sep. 19, 2001

Related U.S. Application Data
(63) Continuation of application No. PCT/NL00/00180, filed on Mar. 17, 2000.

(30) Foreign Application Priority Data
Mar. 19, 1999 (EP) 99200865.6

(51) Int. Cl. A61F 2/08

(52) U.S. Cl. 623/14.13; 435/396

(57) ABSTRACT

Novel scaffolds for tissue engineering muscle are provided that include copolymers of a polyalkylene glycol and an aromatic polyester in the form of a matrix. The scaffold can include autologous muscle cells and/or stem cells.

The invention relates to a scaffold for tissue engineering muscle tissue.

Human muscle tissue has a limited capacity for regeneration. Cardiac muscle tissue even lacks this capacity completely. Consequently, patients suffering from muscle injuries or muscle disorders need quite a long time before they are recovered, if recovery is at all possible.

In the past, it has been postulated that it should be possible to transplant skeletal or cardiac muscle tissue. This would specifically be desirable for patients undergoing plastic or reconstructive surgery, or suffering from muscle dystrophy or heart disease (i.e. cases where recovery of propagation of electrophysiological pulse around a non-functional area is required). Currently, transplantation of autologous or allogenic tissue is employed. However, a drawback of these methods is foremost that the required tissue is extremely scarce, and thus often unavailable when needed. Furthermore, the use of allogenic tissue is associated with a significant risk of infections and the need of powerful immunosuppressant drugs.

In U.S. Pat. No. 5,759,830, it has been proposed that a three-dimensional fibrous scaffold may be used for culturing cells on its surface for producing vascularized tissue in vivo. In accordance with this approach, autologous cells are cultured on a scaffold in vitro. The scaffold having cultured autologous cells attached thereto can subsequently be implanted in a patient having a specific disorder in order to achieve tissue regeneration. After implantation, the attached, pre-cultured cells regenerate the missing or defective tissue, while the scaffold is degraded. In the US patent is stated that these scaffolds can be used for various cell types, among which blood vessel cells, various organ tissue cells, epithelial cells, nerve cells, and muscle cells.

It is indicated that a key point in the configuration of the scaffolds is, that diffusion is enabled to its utmost extent. As the scaffold with attached cells is implanted in non-vascularized form, it is essential that nutrition can be supplied through diffusion. For this reason, it is stated that the scaffolds must have sufficient surface area and exposure to nutrients such that cellular growth and differentiation can occur prior to the ingrowth of blood vessels following implantation. In fact, the only configuration of the scaffolds that is believed to be suitable in accordance with the above US patent, is a fibrous configuration. The open space in between the fibers is believed to be essential in facilitating diffusion to a sufficient degree.

The materials used for making the scaffold described in the US patent are bioabsorbable, biodegradable, synthetic polymers. The only examples mentioned of suitable polymers are polyanhydrides, polyorthoesters, and polyglycolic acids.

The polymers proposed for use as a scaffold material in U.S. Pat. No. 5,759,830 have been found to not have sufficient elastomeric properties to be used as a scaffold for muscle tissue. They do not, or to an insufficient extent, allow for cultivation comprising dynamic or static elongation. Further, they do not mimic the elastomeric properties of the target tissue and do not have the capacity to contribute to the biomechanical properties of the implant in vivo. Also, these materials show a rather fast biodegradation, which is associated with an increased risk of inflammatory response of the patient, as well as an unacceptable reduction in pH of the surroundings of the implant.

It is an object of the present invention to provide an improved scaffold for muscle tissue engineering and to thereby overcome the problems associated with the scaffolds described in the prior art. Specifically, it is desired to provide a scaffold having elastomeric properties closely mimicking the properties of the target tissue. Further, the scaffold should have an optimal biodegradation profile. It should degrade slow enough to lead to sufficient contribution to biomechanical properties after implantation and desirably to prevent formation of scar tissue, and it should degrade fast enough to be resorbed when mechanical strength is no longer needed. In addition, the scaffold must be highly prone to cell attachment, in particular of muscle cells.

Surprisingly, it has been found that the above objects are achieved by using a scaffold based on a highly specific copolymer material. This material is a copolymer of a polyalkylene glycol and an aromatic polyester. Accordingly, the invention relates to the use of a matrix of a copolymer of a polyalkylene glycol and an aromatic polyester as a scaffold for tissue engineering muscle.

It has been found that a scaffold according to the invention does not necessarily have to be fibrous in structure for enabling a sufficient degree of diffusion before in vivo vascularization has taken place. In an aqueous environment, the copolymer on which the scaffold is based forms a hydrogel. Surprisingly, this hydrogel is permeable for nutrient transport to the cells and transport of waste products from the cells. Advantageously, the material on which the present scaffold is based is biocompatible and shows a highly favorable cell attachment. An additional advantage of the present scaffold is related to its mechanical properties. As the material on which the scaffold is based matches the elastomeric properties of muscle tissue, implantation of a cell-scaffold construct is possible at a early timepoint, i.e. after a relatively short period of cultivation in vitro.

As has been mentioned, the material on which the present scaffold is based, is a copolymer of a polyalkylene glycol and an aromatic polyester. Preferably, the copolymer comprises 40-80 wt. %, more preferably 60-70 wt. % of the polyalkylene glycol, and 60-20 wt. %, more preferably 40-30 wt. % of the aromatic polyester. A preferred type of copolymers according to the invention is formed by the group of block copolymers.

Preferably, the polyalkylene glycol has a weight average molecular weight of about 150 to about 4000, more preferably of about 200 to about 1500. The aromatic polyester preferably has a molecular weight of from about 200 to about 5000, more preferably from about 250 to about 4000. The weight average molecular weight of the copolymer preferably lies between about 20,000 and 200,000, more preferably between about 50,000 and about 120,000. The weight average molecular weight may suitably be determined by gel permeation chromatography (GPC). This technique, which is known per se, may for instance be performed using tetrahydrofuran as a solvent and polystyrene as external standard.

In a preferred embodiment, the polyalkylene glycol component has units of the formula -OLO-CO-Q-CO-, wherein O represents oxygen, C represents carbon, L is a divalent organic radical remaining after removal of terminal hydroxyl groups from a poly(oxyalkylene)glycol, and Q is a divalent organic radical.

Preferred polyalkylene glycols are chosen from the group of polyethylene glycol, polypropylene glycol, and polybutylene glycol and copolymers thereof, such as poloxamers. A highly preferred polyalkylene glycol is polyethylene glycol.

The terms alkylene and polyalkylene generally refer to any isomeric structure, i.e. propylene comprises both 1,2-propylene and 1,3-propylene, butylene comprises 1,2-butylene, 1,3-butylene, 2,3-butylene, 1,2-isobutylene, 1,3-isobutylene and 1,4-isobutylene (tetramethylene) and similarly for higher alkylene homologues. The polyalkylene glycol component is preferably terminated with a dicarboxylic acid residue -CO-Q-CO-, if necessary to provide a coupling to the polyester component. Group Q may be an aromatic group having the same definition as R, or may be an aliphatic group such as ethylene, propylene, butylene and the like.

The polyester component preferably has units -O-E-O-CO-R-CO-, wherein O represents oxygen, C represents carbon, E is a substituted or unsubstituted alkylene or oxydialkylene radical having from 2 to 8 carbon atoms, and R is a substituted or unsubstituted divalent aromatic radical.

In a preferred embodiment, the polyester is chosen from the group of polyethylene terephtalate, polypropylene terephtalate, and polybutylene terephtalate. A highly preferred polyester is polybutylene terephtalate.

In a highly preferred embodiment, a copolymer is used which comprises 55 wt. % polyethylene glycol and 45 wt. % polybutylene terephtalate, wherein the molecular weight of the polyethylene glycol is 300. It was found that on this material, both myoblast attachment, proliferation as well as differentiation into myotubes is optimal.

The preparation of the copolymer will now be explained by way of example for a polyethylene glycol/polybutylene terephtalate copolymer. Based on this description, the skilled person will be able to prepare any desired copolymer within the above described class. An alternative manner for preparing polyalkylene glycol/polyester copolymers is disclosed in U.S. Pat. No. 3,908,201.

A polyethylene glycol/polybutylene terephtalate copolymer may be synthesized from a mixture of dimethyle terephtalate, butanediol (in excess), polyethylene glycol, an antioxidant and a catalyst. The mixture is placed in a reaction vessel and heated to about 180° C., and methanol is distilled as transesterification proceeds. During the transesterification, the ester bond with methyl is replaced with an ester bond with butylene. In this step the polyethyene glycol substantially does not react. After transesterification, the temperature is raised slowly to about 245° C., and a vacuum (finally less than 0.1 mbar) is achieved. The excess butanediol is distilled and a prepolymer of butanediol terephtalate condenses with the polyethylene glycol to form a polyethylene/polybutylene terephtalate copolymer. A terephtalate moiety connects the polyethylene glycol units to the polybutylene terephtalate units of the copolymer and thus such copolymer is sometimes also referred to as a polyethylene glycol terephtalate/polybutylene terephtalate copolymer (PEGT/PBT copolymer).

The copolymer described above may conveniently be processed into a desired shape using any known manner such as injection molding, extrusion, and so forth. The objective shape and size will depend on the envisaged application of the scaffold, in particular on the shape and size of the defect in muscle tissue which is intended to be repaired using the present scaffold.

The copolymer may either be formed into a porous or into a fibrous scaffold, thus enabling optimal diffusion of nutrients and waste products. Porous structures may be obtained by techniques known per se, such as salt leaching or sintering. Fibrous structures can inter alia be obtained by extrusion. Preferably, the copolymer is formed into a fibrous structure, as this type of structure more closely resembles natural muscle tissue.

A scaffold based on the above described copolymer, which is of course also encompassed by the present invention, is particularly suitable for use in tissue engineering muscle tissue. In this application, the scaffold may be implanted with or without cells attached thereto.

When the scaffold is implanted without cells attached thereto, the surrounding, healthy muscle tissue will regenerate cells which grow into the scaffold, or cells may migrate from surrounding tissue into the scaffold. This process will be accompanied by vascularization. As the regeneration process proceeds, the scaffold will be slowly broken down. Thus, eventually, the copolymer material of the scaffold will have disappeared and new muscle tissue has been formed.

Preferably, the scaffold is implanted with cells attached thereto. Thus, it is preferred that the scaffold is seeded with cells prior to implantation. The cells may be any type of cells naturally occurring in muscle tissue or any type of cells capable of differentiating into such cells.

Preferred cell types are muscle cells, such as cells forming smooth muscle, cells forming skeletal muscle, or heart muscle cells, stem cells, and satellite cells (stem cells of skeletal muscle tissue). These cells may also be used in their crude form, e.g. in the form of bone marrow, comprising more than one cells type or even extracellular matrix. It is further preferred that the cells are autologous cells, thus minimizing the chance of rejection responses in the patient treated with the present scaffold.

The seeding may be carried out in any known manner, for instance by static seeding. It is preferred, however, that the cells are seeded dynamically as has been described in co-pending European patent application 98203774.9, which is incorporated herein by reference. Although muscle cells require very careful handling and are very difficult to attach to non-natural materials, in accordance with the present invention a good attachment can be obtained.

Subsequent to the seeding process, the cells are preferably cultured in vitro, allowing for a sufficient degree of proliferation and/or differentiation of the cells.

The period required for the culturing may vary broadly and range between one hour and several months, depending on the number of seeded cells and the size of the implant or scaffold.

In this regard, a specific advantage of the scaffold according to the invention is related to the elastomeric properties of the material on which the scaffold is based.

These properties allow to mimic during in vitro cultivation some of the mechanical forces that cells typically experience in vivo, i.e. by stretching the cell-scaffold composite during the tissue engineering in vitro. In particular, the stretching can be carried out in a static manner, i.e. stretching by a specific length per day, or in a dynamic manner whereas the material is stretched and released with a specific frequency. Surprisingly, it was found that stretching increased cellularity, synthesis of extracellular matrix and the expression of a differentiation marker of myocytes. Therefore, cultivation under stretching is the preferred method of engineering muscle tissue.

The invention further relates to the use of the above described scaffold as a medical implant for repairing defective or missing muscle tissue. To give a specific example of the advantages of the invention, it has been found that skeletal muscle may advantageously be substituted with cardiac muscle tissue, which has been tissue engineered in accordance with the invention.

The invention will now be elucidated by the following, non-restrictive examples.

### EXAMPLES

### Example 1

Fabrication and Composition of PEGT/PBT Films

Five different compositions of PEGT/PBT (trade name Polyactive™) (with a PEGT/PBT weight ratio of 40/60, 55/45, 60/40, and 70/30) and 2 different molecular weights of PEG (with a PEG molecular weight of 300 and 1000 Da) were used. The different formulations of Polyactive™ are indicated as: ₐPEGT_{b}/PBT_{c}, in which a is the PEG molecular weight, b is the wt. % of PEG-soft segments and c is the wt. % of PBT-hard segments. Dense substrates (approximately 100 μm thick) were cast from a Polyactive™/chloroform solution on ethanol cleaned glass plates using an in-height adjustable film applicator. The films were then stripped from the glass plates, and were dried in room temperature, and in a vacuum oven at 50° C. overnight. All dense films were used after steam sterilization (121°C., 30 min).

Cell Isolation and Culture

A mouse myogenic cell line C2C12 (ATCC CRL 1772), was maintained as proliferating myoblasts in growth medium (DMEM supplemented with 10% FBS, 10% calf serum (CS), 10 mM HEPES, 2 mM L-glutamine, 100 units/ml penicillin-streptomycin). The myoblasts were cultured in growth media inside T175 culture flasks until 70% confluency was achieved. For harvesting the cells, cultures were washed 3 times in phosphate buffered saline, then incubated for 5 min with a 0.05% trypsin-EDTA solution, followed by addition of an equal amount of growth media and centrifugation for 10 min at 1000 rpm. The cell number was determined using a hematocytomer.

Cell cultures were grown at 37° C. in a humidified 5% CO₂/95% air atmosphere, and media were changed 100% every second day.

Cell Seeding

Dry dense Polyactive™ films were placed inside non-tissue culture-treated 6 well plates and incubated with sterile dH₂O to induce swelling, due to the hydrogel nature of the PEG segment. Equilibrium swelling was achieved after 24 hrs incubation. The increase in surface area was taken into account for the calculation of the cell number during seeding. The seeding density for C2C12 cells was 5,500 cells/cm² for all studies. A concentrated cell suspension (500 μl per film) was added on each film. In addition, cells were seeded on tissue culture polystyrene (TCPS) at the same density as for Polyactive™.

Cell Attachment and Proliferation

To monitor cell attachment on films, the number of attached cells was determined after seeding. To remove the non-attached cells, cultures were washed 3 times with PBS. Attached cells were incubated with 0.05% trypsin-EDTA solution for 5 min, followed by addition of an equal volume of growth media. The cell number was determined using a hematocytomer.

To monitor cell proliferation on films, the number of cells was determined using the same protocol as above.

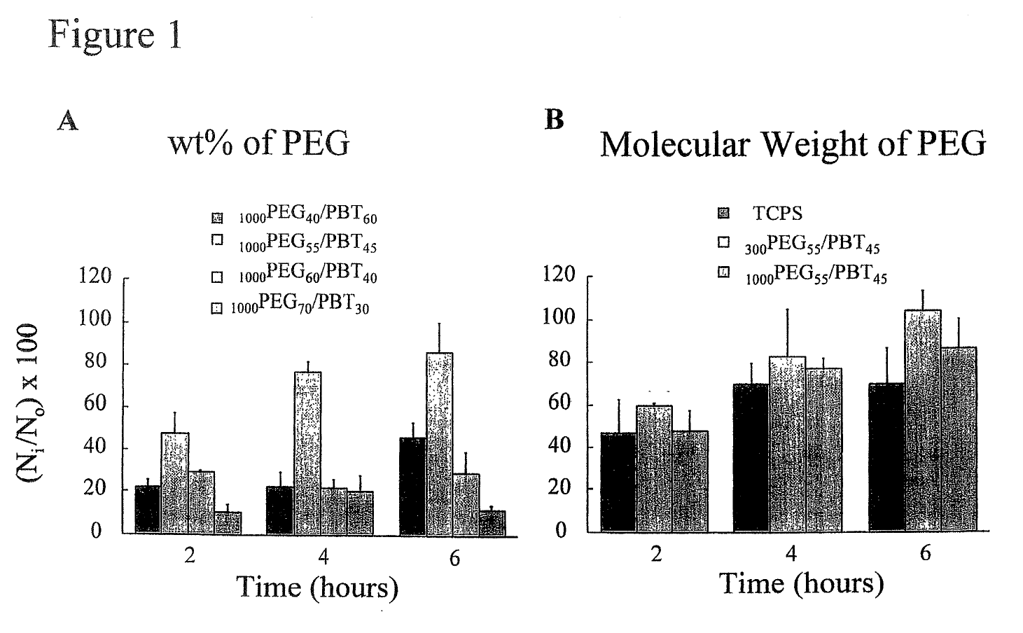

The dynamics of cell attachment were similar when PEGT/PBT films with PEG molecular weights of 300 (₃₀₀PEG₅₅/PBT45) and 1000 Da (₁₀₀₀PEGT₅₅/PBT₄₅) were tested (FIG. 1) In addition, cell attachment on both ₃₀₀PEGT₅₅/PBT₄₅ and ₁₀₀₀PEGT₅₅/PBT₄₅ was similar to TCPS. The percentage of attached cells on ₃₀₀PEGT₅₅/PBT₄₅ increased significantly with time, reaching almost 100% of the seeded cells at 6 hrs of seeding.

Cell proliferation on different PEGT/PBT films with different composition and molecular weight of PEG was studied at 4, 6, 8 and 10 days of cultivation.

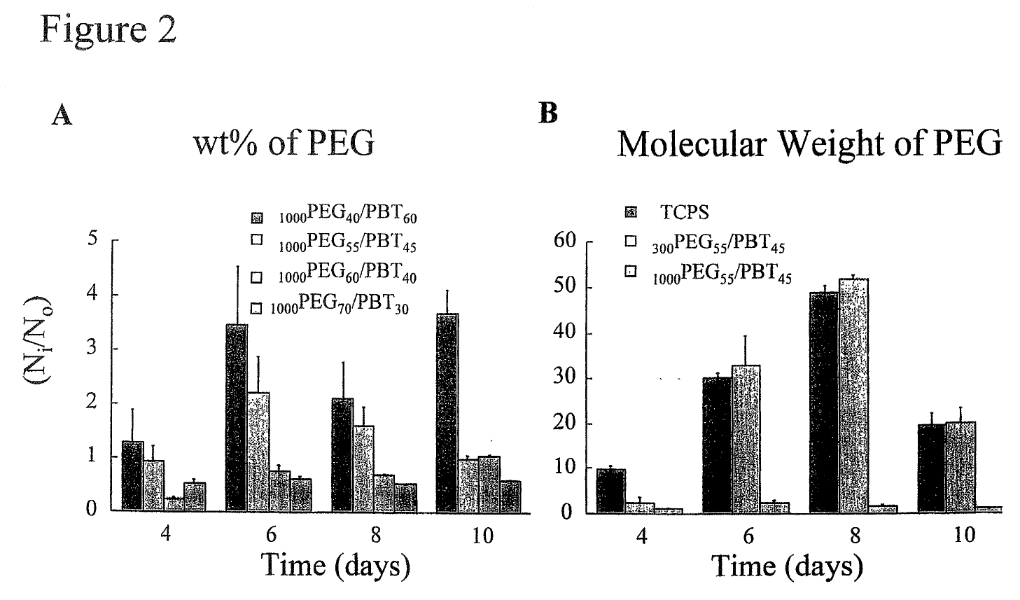

The combination of a PEG molecular weight of 300 Da and a PEGT/PBT composition of 55/45 (₃₀₀PEGT/₅₅/PBT₄₅) resulted in an exponential increase in skeletal muscle cell number of the seeded cells at 10 days of cultivation, in contrast to ₁₀₀₀PEGT₅₅/PBT₄₅ where only modest increases were observed, (FIG. 2). Furthermore, the cell number on the ₃₀₀PEGT₅₅/PBT₄₅ was similar to TCPS at all time points examined.

Differentiated Function

Myotube formation: To induce fusion of myoblasts into myotubes, 1 week cultures were switched to differentiation media (DMEM supplemented with 2% horse serum, 10 mM HEPES, 2 mM L-glutamine and 100 units/ml penicillin G). Cultures remained in differentiation media for 3-5 days and then the presence of myotubes was visualized with light microscopy.

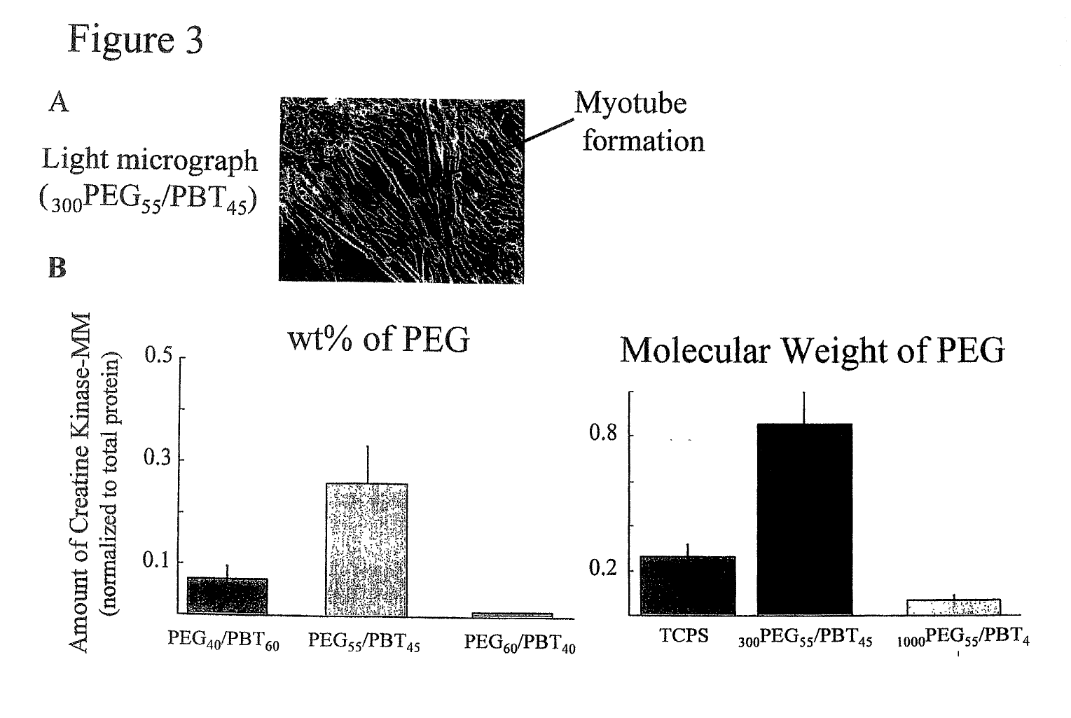

After 3 days incubation with differentiation media the majority of cells were multinucleated myotubes with lengths longer than 60 μm (FIG. 3A). No visible formation of myotubes was observed when other PEGT/PBT formulations were used.

Western Blot analysis: Samples were rinsed twice in PBS and total cell lysates were harvested in a PBS buffer containing a cocktail of protease inhibitors for 1 min. After centrifugation for 10 min at 12,000 g at 4° C., samples were stored at −80° C. Total protein was measured by a commercially available kit (Biorad). Homogenates were diluted (1 part sample to 2 parts buffer), in Tricine buffer containing 5% mercaptoethanol, and boiled for 5 min. Homogenates containing 15 μg of total protein each were separated on 10-20% Tris-Tricine minigels. Purified human CK-MM, was used as positive control for CK-MM.

Eluted proteins were electroblotted. Blots were first incubated overnight at 4° C. with 5% nonfat dry milk in PBS with 0.05% Tween-20 (PBS-T), to block nonspecific binding of antibodies, and then for an additional 1-2 hrs with the appropriate primary antibody. The primary antibody was a goat anti-CK-MM, diluted 1:2500 in PBS-T. Blots were washed 5 times and incubated for 1 h at room temperature with a rabbit antigoat IgG antibody, conjugated to horseradish peroxidase and diluted 1:3000 in PBS-T. After 5 additional washes, immunocomplexes were developed using enhanced horseradish peroxidase/luminol chemiluminescence and detected after exposure to photographic film for 5-30 sec.

The PEGT/PBT composition that resulted in the highest expression of CK-MM, as compared to ₁₀₀₀PEGT₄₀/PBT₆₀ and ₁₀₀₀PEGT₆₀/PBT₄₀, was the ₁₀₀₀PEGT₅₅/PBT₄₅ (FIG. 3B, left chart). Similar to the proliferation data, the combination of a PEG molecular weight of 300 Da and a PEGT/PBT composition of 55/45 (₃₀₀PEGT₅₅/PBT₄₅) resulted in cultures with expression levels of CK-MM significantly higher than ₁₀₀₀PEGT₅₅/PBT₄₅ and TCPS (FIG. 3).

### Example 2

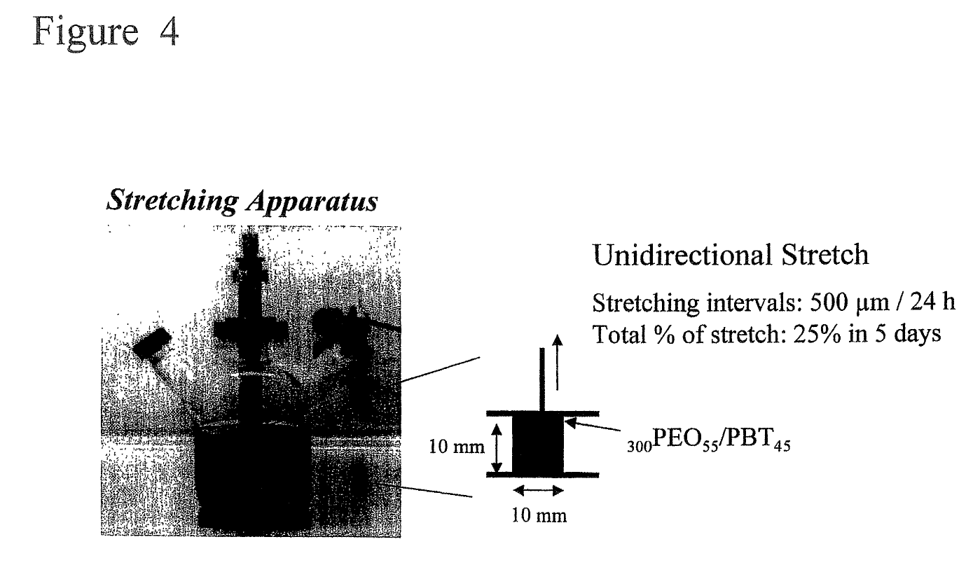

Porous salt leached Polyactive scaffolds (₃₀₀PEG₅₅/PBT₄₅, 300 μm thick, 10 mm diameter) were sterilized using ethylene oxide, equilibrated as described in example 1, fixed to a stretching device as shown in FIG. 4 and seeded with 1 million cells per scaffold in a spinner flask. Whereas in control experiments no stretching was applied, in the stretch group the scaffolds were elongated by 500 μm per 24 h, for up to 5 days. Analysis was as specified in example 1, histology using H&E stain.

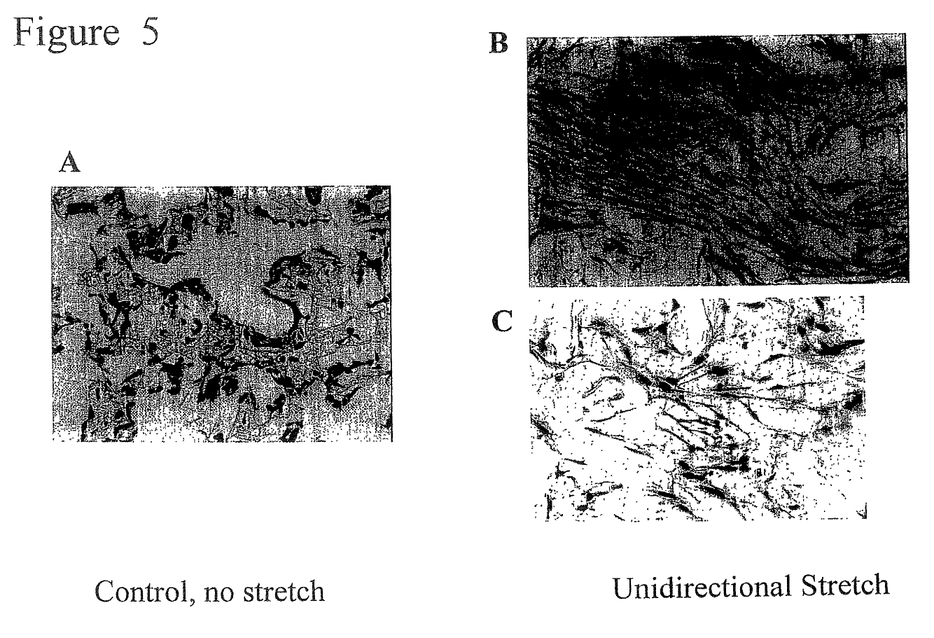

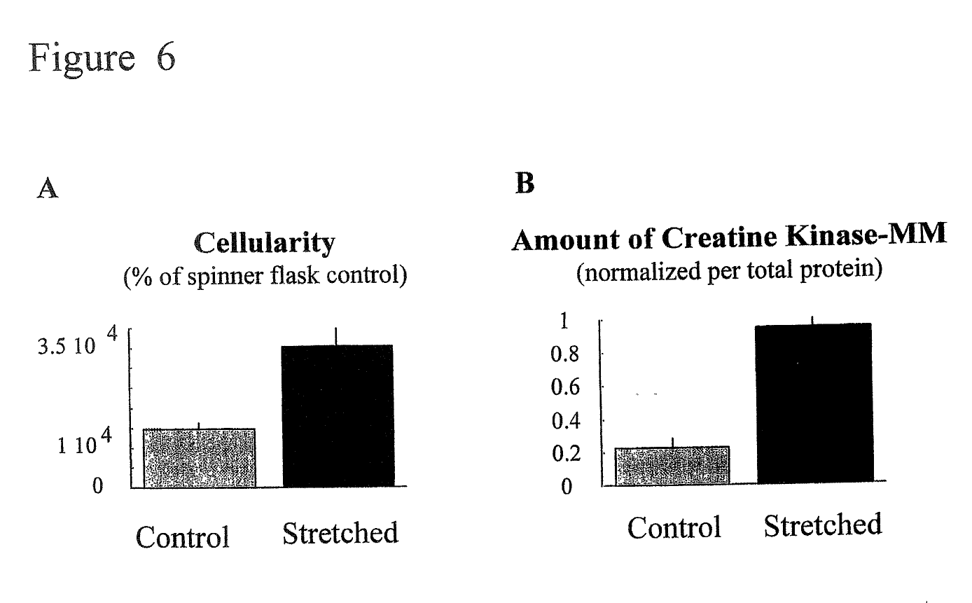

Only in the stretched sample extracellular matrix can be recognized after 7 days in culture (FIG. 5A, C). Furthermore, cells appear more differentiated in the samples cultivated under stretched conditions (FIG. 5A, B). The histological findings are in agreement with the increased cellularity in the starched samples (FIG. 6A) and the elevated amount of kretatine kinase in the stretched group (FIG. 6B).

Figure Legends

FIG. 1: Percent of seeded skeletal muscle cells attached on different PEGT/PBT compositions and molecular weights of PEG with an initial seeding density of 5,500 cell/cm² as a function of time. A) Four different PEGT/PBT wt. % were compared (₁₀₀₀PEG₄₀/PBT₆₀, ₁₀₀₀PEG₅₅/PBT₄₅, ₁₀₀₀PEG₆₀/PBT₄₀ and ₁₀₀₀PEG₇₀/PBT₃₀. B) Two PEG molecular weights (300 and 1000 Da) were compared to TCPS.

FIG. 2: Percent of seeded skeletal muscle cells proliferated on different PEGT/PBT compositions and molecular weights of PEG with an initial seeding density of 5,500 cell/cm² as a function of time. A) Four different PEGT/PBT wt. % were compared (₁₀₀₀PEG₄₀/PBT₆₀, ₁₀₀₀PEG₅₅/PBT₄₅, ₁₀₀₀PEG₆₀/PBT₄₀ and ₁₀₀₀PEG₇₀/PBT₃₀. B) Two PEG molecular weights (300 and 1000 Da) were compared to TCPS.

FIG. 3: Differentiation of skeletal muscle cells on different PEGT/PBT compositions and molecular weights of PEG at 10 days of cultivation. A) Upon switching to differentiation media skeletal muscle cells cultured on ₃₀₀PEG₅₅/PBT₄₅ fused to form myotubes, bar 10 μm. No index of myotube formation was observed when other PEGT/PBT compositions and molecular weights of PEG were used. B) Western blot analysis of cell lysates by laser scanning densitometry for CK-MM. Four different PEGT/PBT wt. % were compared (₁₀₀₀PEG₄₀/PBT₆₀, ₁₀₀₀PEG₅₅/PBT₄₅, ₁₀₀₀PEG₆₀/PBT₄₀ and ₁₀₀₀PEG₇₀/PBT₃₀). Two PEG molecular weights (300 and 1000 Da) were compared to TCPS.

FIG. 4: Scheme of the stretching setup.

FIG. 5: Histology (H&E stain) of A: unstretched and B, C: stretched samples after 7 days of cultivation. ₃₀₀PEG₅₅/PBT₄₅ was used.

FIG. 6: Cellularity (A) and amount of kreatine kinase (B) in stretched and control samples after 7 days of cultivation. ₃₀₀PEG ₅₅/PBT₄₅ was used.

1. The use of a matrix of a copolymer of a polyalkylene glycol and an aromatic polyester as a scaffold for tissue engineering muscle.

2. The use according to claim 1, wherein the copolymer comprises 40-80 wt. %, based on the weight of the copolymer, of the polyalkylene glycol, and 60-20 wt. %, based on the weight of the copolymer, of the aromatic polyester.

3. The use according to claim 1 or 2, wherein the polyalkylene glycol is chosen from the group of polyethylene glycol, polypropylene glycol, and polybutylene glycol.

4. The use according to claim 3, wherein the polyalkylene glycol is polyethylene glycol.

5. The use according to any of the preceding claims, wherein the aromatic polyester is chosen from the group of polethylene terephtalate, polypropylene terephtalate, and polybutylene terephtalate.

6. The use according to claim 5, wherein the aromatic polyester is polybutylene terephtalate.

7. The use according to any of the preceding claims, wherein the scaffold is provided with autologous muscle cells and/or stem cells.

8. A matrix of a copolymer of a polyalkylene glycol and an aromatic polyester to be used as a scaffold for tissue engineering muscle according to any of the preceding claims.

9. A method for repairing muscle tissue comprising implantation of a scaffold according to claim 8.

10. The use of a copolymer of a polyalkylene glycol and an aromatic polyester for the manufacture of a scaffold for implantation in a patient for repairing muscle tissue.

11. A method for manufacturing an implant for muscle tissue, wherein a matrix of a copolymer of a polyalkylene glycol and an aromatic polyester is provided with autologous muscle cells and/or stem cells by immersing the matrix in a culture medium, which culture medium comprises the cells and is kept under dynamic conditions.

12. A method according to claim 11, wherein the cells are cultured under dynamic conditions.

13. A method according to claim 11 or 12, wherein the scaffold is cultivated under stretching.
